(19) United States
(12) Patent Application Publication
(10) Pub. No.: US 2014/0323577 A1
(43) Pub. Date: Oct. 30, 2014

(54) METHODS FOR REDUCING THE RISK OF CONVERSION TO CLINICALLY DEFINITE MULTIPLE SCLEROSIS

(71) Applicant: Sanofi-Aventis U.S. LLC, Bridgewater, NJ (US)

(72) Inventor: Barbara WAMIL, Flanders, NJ (US)

(73) Assignee: Sanofi-Aventis U.S. LLC, Bridgewater, NJ (US)

(21) Appl. No.: 14/260,928

(22) Filed: Apr. 24, 2014

Related U.S. Application Data
(60) Provisional application No. 61/815,982, filed on Apr. 25, 2013.

(51) Int. Cl. A61K 31/277

(52) U.S. Cl. CPC A61K 31/277
USPC 514/626

(57) ABSTRACT

This invention relates to a method for reducing the risk of conversion to clinically definite multiple sclerosis (CDMS) in patients who have experienced their first neurological symptoms consistent with clinically isolated syndrome (CIS).

### FIELD OF THE INVENTION

This invention relates to a method for reducing the risk of conversion to clinically definite multiple sclerosis (CDMS) in patients who have experienced their first neurological symptoms consistent with clinically isolated syndrome (CIS).

### BACKGROUND OF THE INVENTION

Teriflunomide is a novel oral disease-modifying therapy (DMT) for the treatment of relapsing forms of multiple sclerosis (RMS). Teriflunomide blocks de novo pyrimidine synthesis, which inhibits the replication and function of activated (but not resting) lymphocytes.

The compound of (Z)-2-cyano-3-hydroxy-but-2-enoic acid-(4′-trifluoromethylphenyl)-amide (also known as teriflunomide, Formula I) is described in U.S. Pat. No. 5,679,709.

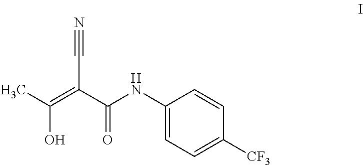 [SMILES: C/C(O)=C(\C#N)C(=O)Nc1ccc(C(F)(F)F)cc1]

The use of teriflunomide for treating multiple sclerosis is described in U.S. Pat. No. 6,794,410.

CIS is defined as a first clinical attack with features suggestive of MS. It typically occurs in young adults and is often a prelude to CDMS, defined as occurrence of a second clinical attack. It is now found that teriflunomide is useful for reducing the risk of conversion to CDMS in patients who have experienced their first neurological symptoms consistent with CIS.

### SUMMARY OF THE INVENTION

Accordingly, this invention relates to a method for reducing the risk of conversion to CDMS comprising administering to a patient who has experienced a first neurological symptom consistent with CIS a pharmaceutically effective amount of teriflunomide or a pharmaceutically acceptable salt thereof.

### DETAILED DESCRIPTION OF THE INVENTION

As used above, and throughout the description of the invention, the following terms, unless otherwise indicated, shall be understood to have the following meanings:

“Patient” means a human.

“Pharmaceutically acceptable salt” refers to the relatively non-toxic, inorganic and organic acid addition salts, and base addition salts, of teriflunomide which can be used in medicinal preparations.

One aspect of the invention is related to a method for reducing the risk of conversion to CDMS comprising administering to a patient who has experienced a first neurological symptom consistent with CIS a pharmaceutically effective amount of teriflunomide.

Another aspect of the invention is related to a method for reducing the risk of conversion to CDMS comprising administering to a patient who has experienced a first neurological symptom consistent with CIS about 7 mg or about 14 mg of teriflunomide daily.

Another aspect of the invention is related to a method for reducing the risk of conversion to CDMS comprising administering to a patient who has experienced a first neurological event consistent with demyelination and MRI lesions characteristic of MS a pharmaceutically effective amount of teriflunomide or a pharmaceutically acceptable salt thereof.

Another aspect of the invention is related to a method for reducing the risk of conversion to CDMS comprising administering to a patient who has experienced a first neurological event consistent with demyelination and MRI lesions characteristic of MS a pharmaceutically effective amount of teriflunomide.

Another aspect of the invention is related to a method for reducing the risk of conversion to CDMS comprising administering to a patient who has experienced a first neurological event consistent with demyelination and MRI lesions characteristic of MS about 7 mg or about 14 mg of teriflunomide daily.

Another aspect of the invention is related to the use of teriflunomide or a pharmaceutically acceptable salt thereof in reducing the risk of conversion to CDMS comprising administering to a patient who has experienced a first neurological symptom consistent with CIS.

Another aspect of the invention is related to the use of teriflunomide in reducing the risk of conversion to CDMS comprising administering to a patient who has experienced a first neurological symptom consistent with CIS.

Another aspect of the invention is related to the use of teriflunomide in reducing the risk of conversion to CDMS comprising administering to a patient who has experienced a first neurological symptom consistent with CIS, wherein teriflunomide is administered to the patient at about 7 mg or 14 mg daily dose.

Another aspect of the invention is related to the use of teriflunomide or a pharmaceutically acceptable salt thereof in reducing the risk of conversion to CDMS comprising administering to a patient who has experienced a first neurological event consistent with demyelination and MRI lesions characteristic of MS.

Another aspect of the invention is related to the use of teriflunomide in reducing the risk of conversion to CDMS comprising administering to a patient who has experienced a first neurological event consistent with demyelination and MRI lesions characteristic of MS.

Another aspect of the invention is related to the use of teriflunomide in reducing the risk of conversion to CDMS comprising administering to a patient who has experienced a first neurological event consistent with demyelination and MRI lesions characteristic of MS, wherein teriflunomide is administered to the patient at about 7 mg or 14 mg daily dose.

The present invention may be better understood by reference to the following non-limiting Example, which is exemplary of the invention. It should in no way be construed, however, as limiting the breath of the scope of the invention.

### Example

An international, multi-center, randomized, double-blind, placebo-controlled, parallel group study to evaluate the efficacy and safety of two year treatment with teriflunomide 7 mg once daily and 14 mg once daily versus placebo in patients with a first clinical episode suggestive of multiple sclerosis plus a long term extension period was conducted.

Objective: The primary objective of the study was to demonstrate the effect of teriflunomide (14 mg/day and 7 mg/day) compared to placebo for reducing conversion of patients presenting with their first clinical episode consistent with MS to CDMS as defined by the occurrence of a second clinical relapse.

Methodology: This was a multi-center, multinational, randomized, placebo-controlled, double blind, parallel group (3 groups) study stratified by center and baseline monofocal/multifocal status. After a screening phase of up to 4 weeks, the patients were randomized (1:1:1) to 1 of 3 treatment groups (placebo, 7 mg teriflunomide or 14 mg teriflunomide once daily) and treated double-blind for 108 weeks for those who did not experience conversion to CDMS, and at least 24 weeks for patients who experienced conversion to CDMS.

Diagnosis and criteria for inclusion: Patients with a first acute or subacute, well-defined neurological event consistent with demyelination with onset of MS symptoms occurring within 90 days of randomization; and abnormal MRI with 2 or more T2 lesions at least 3 mm in diameter that are characteristic of MS.

Duration of treatment: Up to 108 weeks for patients who do not experience conversion to CDMS

Duration of observation: Up to 116 weeks (screening period up to 4 weeks, 108 weeks of treatment period and 4 week rapid elimination period)

### Criteria for Evaluation:

Efficacy/pharmacodynamics: The primary efficacy variable was time to CDMS defined by the occurrence of a relapse: new neurological abnormality separated by at least 30 days from onset of a preceding clinical event, present for at least 24 hours and occurring in the absence of fever or known infection.

The key secondary efficacy variable was time to conversion to definite MS as demonstrated by dissemination of MRI lesions in time (as per the 2005 revised McDonald criteria) or a relapse (as defined for the primary endpoint), whichever occurs first: defined through detection of gadolinium enhancement at least 3 months after the onset of the initial clinical event, and detection of a new T2 lesion if it appeared at any time compared with a reference scan done at least 30 days after the onset of the initial clinical event.

Statistical methods: Analysis population: Efficacy analyses were based on the intent-to-treat (ITT) population consisting of all randomized patients, as randomized, who had at least 1 day exposure to study medication.

Efficacy analysis: The primary analysis of time to conversion to CDMS was performed using Cox regression model with treatment, region, and baseline monofocal/multifocal status as independent variables. The assessment of efficacy was based on (pairwise) hazard rate ratio comparisons of each active dose of teriflunomide versus placebo. Two-sided 95% confidence intervals of the hazard rates ratio were provided for each teriflunomide treatment effect estimate versus placebo. The log-rank test was provided as supportive.

The key secondary endpoint was analyzed using the same Cox regression model. ARR was analyzed using a Poisson regression model with robust error variance including the number of confirmed relapses with their onset between randomization date and last dose date as the response variable, treatment, region, and baseline monofocal/multifocal status as covariates, and log transformed standardized exposure duration as an offset variable.

Multiplicity procedure: Step down approach was used for multiplicity adjustment for 2 doses of teriflunomide and the primary and key secondary endpoints. Treatment comparison of teriflunomide 14 mg vs. placebo in time to conversion to CDMS was tested at 5% significance level. If significant, comparison of teriflunomide 7 mg vs. placebo in time to conversion to CDMS was tested at 5% significance level. If significant, comparison of teriflunomide 14 mg vs. placebo for the key secondary endpoint was tested at 5% significance level. If significant, comparison of teriflunomide 7 mg vs. placebo for the key secondary endpoint was tested at 5% significance level.

### Summary:

Population characteristics: A total of 618 patients were randomized in the study, of which 614 (99.4%) were exposed to study medication and included in the intent-to-treat (ITT) population. The summary of analysis population is presented in Table I.

TABLE 1
Analysis populations - Randomized population

| | | teriflunomide | |
|---|---|---|---|
| | Placebo | 7 mg | 14 mg |
| Randomized population | 197 (100%) | 205 (100%) | 216 (100%) |
| Efficacy population | | | |
| ITT population | 197 (100%) | 203 (99.0%) | 214 (99.1%) |
| PP population | 187 (94.9%) | 198 (96.6%) | 209 (96.8%) |
| PK population | 190 | 206 | 216 |
| Safety population | 191 | 207 | 216 |

Note:
The safety and PK patients are tabulated according to treatment actually received (as treated). For the other populations, patients are tabulated according to their randomized treatment.

454 (73.9%) patients completed 108 weeks of study treatment or entered the extension: 71.6% in the placebo group, 73.2% in the teriflunomide 7 mg group, and 75.5% in the teriflunomide 14 mg group. The most common reason for treatment discontinuation among patients not entering the extension was adverse events with a discontinuation rate of 9.1%, 12.2%, and 8.3% in placebo, teriflunomide 7 mg and teriflunomide 14 mg groups, respectively. The mean (±SD) age was 32.1 (±8.5) years and the study population was primarily Caucasian (96.1%).

TABLE 2
Patient disposition with patients who entered the extension
considered as completed - Randomized population

| | | | teriflunomide | | | |
|---|---|---|---|---|---|---|
| | Placebo | | 7 mg | | 14 mg | |
| | (N = 197) | | (N = 205) | | (N = 216) | |
| Randomized and not treated^{a} | 0 | | 2 | (1.0%) | 2 | (0.9%) |
| Randomized and treated | 197 | (100%) | 203 | (99.0%) | 214 | (99.1%) |
| Completed study treatment period^{b} | 141 | (71.6%) | 150 | (73.2%) | 163 | (75.5%) |
| Completed 108 weeks of | 75 | (38.1%) | 78 | (38.0%) | 96 | (44.4%) |
| treatment | | | | | | |
| Entered the extension | 66 | (33.5%) | 72 | (35.1%) | 67 | (31.0%) |
| Did not complete study treatment | 56 | (28.4%) | 53 | (25.9%) | 51 | (23.6%) |
| period | | | | | | |
| Reason for study treatment | | | | | | |
| discontinuation | | | | | | |
| Adverse event | 18 | (9.1%) | 25 | (12.2%) | 18 | (8.3%) |
| Lack of efficacy | 19 | (9.6%) | 6 | (2.9%) | 12 | (5.6%) |
| Poor compliance to protocol | 0 | | 0 | | 0 | |
| Lost to follow-up | 1 | (0.5%) | 1 | (0.5%) | 1 | (0.5%) |
| Death | 1 | (0.5%) | 0 | | 0 | |
| Progressive Disease | 3 | (1.5%) | 1 | (0.5%) | 0 | |
| Subject did not wish to continue | 12 | (6.1%) | 18 | (8.8%) | 15 | (6.9%) |
| Other reason | 2 | (1.0%) | 2 | (1.0%) | 5 | (2.3%) |

^{a}Includes 3 patients that entered the extension directly from the screening phase
^{b}Defined as completing 108 weeks of treatment or entering the extension period
Note:
Percentages are calculated using the number of randomized patients as denominator.

The baseline disease characteristics for all randomized patients were generally similar among treatment groups. The median time since neurological event was 2 months and the median time since clinically isolated syndrome (CIS) evaluation was 1 month. 59.1% of the patients entered the study with a monofocal episode.

TABLE 3
Baseline disease characteristics - Randomized population

| | | | teriflunomide | | | | | |
|---|---|---|---|---|---|---|---|---|
| | Placebo | | 7 mg | | 14 mg | | All | |
| | (N = 197) | | (N = 205) | | (N = 216) | | (N = 618) | |
| Time since neurological event (months) | | | | | | | | |
| Number | 197 | | 205 | | 216 | | 618 | |
| Mean (SD) | 1.88 | (0.52) | 1.89 | (0.56) | 1.80 | (0.56) | 1.85 | (0.55) |
| Median | 2.00 | | 2.00 | | 2.00 | | 2.00 | |
| Min:Max | 1.0:3.0 | | 1.0:3.0 | | 1.0:3.0 | | 1.0:3.0 | |
| Time since CIS evaluation (month) | | | | | | | | |
| Number | 197 | | 205 | | 216 | | 618 | |
| Mean (SD) | 1.39 | (0.62) | 1.50 | (0.63) | 1.41 | (0.65) | 1.43 | (0.63) |
| Median | 1.00 | | 1.00 | | 1.00 | | 1.00 | |
| Min:Max | 0.0:3.0 | | 0.0:3.0 | | 0.0:3.0 | | 0.0:3.0 | |
| Randomized Baseline status | | | | | | | | |
| Number | 197 | | 205 | | 216 | | 618 | |
| Monofocal | 117 | (59.4%) | 125 | (61.0%) | 128 | (59.3%) | 370 | (59.9%) |
| Multifocal | 80 | (40.6%) | 80 | (39.0%) | 88 | (40.7%) | 248 | (40.1%) |
| Actual Baseline status | | | | | | | | |
| Number | 197 | | 205 | | 216 | | 618 | |
| Monofocal | 114 | (57.9%) | 122 | (59.5%) | 129 | (59.7%) | 365 | (59.1%) |
| Multifocal | 83 | (42.1%) | 83 | (40.5%) | 87 | (40.3%) | 253 | (40.9%) |
| Baseline EDSS score | | | | | | | | |
| Number | 196 | | 205 | | 215 | | 616 | |
| Mean (SD) | 1.71 | (1.00) | 1.50 | (1.02) | 1.80 | (0.97) | 1.67 | (1.00) |
| Median | 1.50 | | 1.50 | | 2.00 | | 1.50 | |
| Min:Max | 0.0:5.5 | | 0.0:6.0 | | 0.0:5.0 | | 0.0:6.0 | |
| Number of baseline Gadolinium-enhancing | | | | | | | | |
| lesions | | | | | | | | |
| Number | 177 | | 187 | | 199 | | 563 | |
| Mean (SD) | 1.4 | (4.1) | 1.1 | (3.0) | 1.3 | (3.7) | 1.3 | (3.6) |
| Median | 0.0 | | 0.0 | | 0.0 | | 0.0 | |
| Min:Max | 0:35 | | 0:30 | | 0:31 | | 0:35 | |
| 0 | 119 | (60.4%) | 121 | (59.0%) | 129 | (59.7%) | 369 | (59.7%) |
| ≧1 | 58 | (29.4%) | 66 | (32.2%) | 70 | (32.4%) | 194 | (31.4%) |
| Baseline burden of disease (ml) | | | | | | | | |
| Number | 177 | | 187 | | 200 | | 564 | |
| Mean (SD) | 9.15 | (10.69) | 8.07 | (9.98) | 8.78 | (9.36) | 8.66 | (9.99) |

| | | | | |
|---|---|---|---|---|
| Median | 5.33 | 4.94 | 5.32 | 5.26 |
| Min:Max | 0.3:73.0 | 0.2:67.1 | 0.3:50.7 | 0.2:73.0 |

Efficacy results: Teriflunomide 14 mg significantly reduced the risk of conversion to CDMS compared to placebo by 42.6% (p=0.0087) while 7 mg reduced the risk by 37.2% (p=0.0271) compared to placebo. The estimated cumulative rate of conversion to CDMS in the placebo, teriflunomide 7 mg, and teriflunomide 14 mg, respectively, was 35.9%, 27.6% and 24.0% at 108 weeks.

TABLE 4
Analysis of time to conversion to CDMS - ITT population

| | | teriflunomide | |
|---|---|---|---|
| | Placebo | 7 mg | 14 mg |
| | (N = 197) | (N = 203) | (N = 214) |
| Number of patients with | 55 (27.9%) | 39 (19.2%) | 38 (17.8%) |
| conversion to CDMS | | | |
| Number of patients who were censored | 142 (72.1%) | 164 (80.8%) | 176 (82.2%) |
| Kaplan-Meier estimates of probability | | | |
| of conversion (95% CI) at^{a} | | | |
| 24 weeks | 0.143 (0.092, | 0.087 (0.046, | 0.090 (0.050, |
| | 0.194) | 0.128) | 0.130) |
| 48 weeks | 0.260 (0.192, | 0.142 (0.089, | 0.137 (0.086, |
| | 0.328) | 0.196) | 0.187) |
| 108 weeks | 0.359 (0.278, | 0.276 (0.199, | 0.240 (0.170, |
| | 0.439) | 0.354) | 0.310) |
| Hazard ratio (95% CI)^{b} | | 0.628 (0.416, | 0.574 (0.379, |
| | | 0.949) | 0.869) |
| P-value^{c} | | 0.0271 | 0.0087 |
| Log-rank P-value ^{d} | | 0.0422 | 0.0133 |

^{a}Derived from Kapan-Meier estimates
^{b}Derived using Cox proportional hazard model with treatment, region and baseline monofocal/multifocal status as covariates.
^{c}Derived using Wald chi-squared test in the Cox proportional hazard model
^{d} Derived from log-rank test with stratification of region and baseline monofocal/multifocal status

Teriflunomide 14 mg significantly reduced the risk of conversion to DMS as demonstrated by dissemination of MRI lesions in time or a relapse by 34.9% (p=0.0003) while 7 mg reduced the risk by 31.4% (p=0.0020) compared to placebo. The estimated cumulative rate of conversion to DMS (dissemination of MRI lesions in time or confirmed relapse) in the placebo, teriflunomide 7 mg, and teriflunomide 14 mg, respectively, was 87.0%, 73.3% and 71.5% at 108 weeks.

TABLE 5
Analysis of time to conversion to DMS as demonstrated by dissemination
of MRI lesions in time or a relapse - ITT population

| | | teriflunomide | |
|---|---|---|---|
| | Placebo | 7 mg | 14 mg |
| | (N = 197) | (N = 203) | (N = 214) |
| Number of patients with | 149 (75.6%) | 125 (61.6%) | 136 (63.6%) |
| conversion to DMS | | | |
| Relapse | 26 (13.2%) | 15 (7.4%) | 25 (11.7%) |
| Dissemination of MRI lesion in time | 123 (62.4%) | 110 (54.2%) | 111 (51.9%) |
| Number of patients who were censored | 48 (24.4%) | 78 (38.4%) | 78 (36.4%) |
| Kaplan-Meier estimates of probability | | | |
| of conversion (95% CI) at^{a} | | | |
| 24 weeks | 0.582 (0.510, | 0.457 (0.385, | 0.460 (0.390, |
| | 0.654) | 0.529) | 0.530) |
| 48 weeks | 0.724 (0.657, | 0.573 (0.498, | 0.578 (0.506, |
| | 0.791) | 0.647) | 0.649) |
| 108 weeks | 0.870 (0.812, | 0.733 (0.660, | 0.715 (0.645, |
| | 0.927) | 0.807) | 0.784) |
| Hazard ratio (95% CI)^{b} | | 0.686 (0.540, | 0.651 (0.515, |
| | | 0.871) | 0.822) |
| P-value^{c} | | 0.0020 | 0.0003 |
| Log-rank P-value ^{d} | | 0.0028 | 0.0002 |

^{a}Derived from Kapan-Meier estimates
^{b}Derived using Cox proportional hazard model with treatment, region and baseline monofocal/multifocal status as covariates.
^{c}Derived using Wald chi-squared test in the Cox proportional hazard model
^{d} Derived from log-rank test with stratification of region and baseline monofocal/multifocal status

The on-treatment adjusted ARR was 0.284 in the placebo group, 0.190 in the teriflunomide 7 mg group, and 0.194 in the teriflunomide 14 mg group. This corresponded to a non-statistically significant risk reduction of 31.9% (p=0.0579) in the teriflunomide 14 mg group and 33.1% (p=0.0541) in the teriflunomide 7 mg group relative to placebo.

TABLE 6
Analysis of confirmed MS relapse - ITT population

| | | teriflunomide | |
|---|---|---|---|
| | Placebo | 7 mg | 14 mg |
| | (N = 197) | (N = 203) | (N = 214) |
| Number of patients | | | |
| with ≧1 relapses | | | |
| Yes | 58 (29.4%) | 42 (20.7%) | 45 (21.0%) |
| No | 139 (70.6%) | 161 (79.3%) | 169 (79.0%) |
| Number of relapses | | | |
| 0 | 139 (70.6%) | 161 (79.3%) | 169 (79.0%) |
| 1 | 42 (21.3%) | 33 (16.3%) | 33 (15.4%) |
| 2 | 13 (6.6%) | 7 (3.4%) | 10 (4.7%) |
| >=3 | 3 (1.5%) | 2 (1.0%) | 2 (0.9%) |
| Total number of relapses | 77 | 54 | 60 |
| Total patient-years followed | 249.4 | 258.5 | 288.9 |
| Unadjusted annualized | 0.309 | 0.209 | 0.208 |
| relapses rate ^{a} | | | |
| Adjusted annualized | | | |
| relapse rate ^{b} | | | |
| Estimate (95% CI) | 0.284 (0.214, | 0.190 (0.139, | 0.194 (0.143, |
| | 0.378) | 0.260) | 0.263) |
| Relative risk (95% CI) | | 0.669 (0.444, | 0.681 (0.458, |
| | | 1.007) | 1.013) |
| P-value^{c} | | 0.0541 | 0.0579 |
| | | −0.094 (−0.191, | −0.091 (−0.186, |
| Risk difference (95% CI) | | 0.003) | 0.005) |
| P-value^{d} | | 0.0576 | 0.0628 |

### CONCLUSIONS

Teriflunomide 14 mg significantly reduced the risk of conversion to CDMS compared to placebo by 42.6% (p=0.0087) while 7 mg reduced the risk by 37.2% (p=0.0271) compared to placebo. The risk of conversion to DMS as demonstrated by dissemination of MRI lesions in time or a relapse was significantly reduced by 34.9% (p=0.0003) in the teriflunomide 14 mg group and 31.4% (p=0.0020) in the teriflunomide 7 mg group compared to placebo. The on-treatment adjusted ARR was non-significantly reduced by 31.9% (p=0.0579) in the 14 mg group and 33.1% (p=0.0541) in the 7 mg group relative to placebo. Teriflunomide was well tolerated with a manageable safety profile on both doses.

We claim:

1. A method for reducing the risk of conversion to CDMS comprising administering to a patient who has experienced a first neurological symptom consistent with CIS a pharmaceutically effective amount of teriflunomide or a pharmaceutically acceptable salt thereof.

2. The method according to claim 1, comprising administering to the patient a pharmaceutically effective amount of teriflunomide.

3. The method according to claim 2, comprising administering to the patient about 7 mg or about 14 mg of teriflunomide daily.

4. A method for reducing the risk of conversion to CDMS comprising administering to a patient who has experienced a first neurological event consistent with demyelination and MRI lesions characteristic of MS a pharmaceutically effective amount of teriflunomide or a pharmaceutically acceptable salt thereof.

5. The method according to claim 4, comprising administering to the patient a pharmaceutically effective amount of teriflunomide.

6. The method according to claim 5, comprising administering to the patient about 7 mg or about 14 mg of teriflunomide daily.
